(19) United States
(12) Patent Application Publication
(10) Pub. No.: US 2013/0045273 A1
(43) Pub. Date: Feb. 21, 2013

(54) METHODS FOR USING NUTRITIONAL SUPPLEMENTS CONTAINING LIPOIC ACIDS AND SULFUR CONTAINING COMPOUNDS

(71) Applicants: John Cuomo, Salt Lake City, UT (US); Mark J. Brown, Salt Lake City, UT (US); Brian M. Dixon, Salt Lake City, UT (US); Christine Dern, Salt Lake City, UT (US)

(72) Inventors: John Cuomo, Salt Lake City, UT (US); Mark J. Brown, Salt Lake City, UT (US); Brian M. Dixon, Salt Lake City, UT (US); Adam Dern, Salt Lake City, UT (US)

(21) Appl. No.: 13/213,313

(22) Filed: Aug. 19, 2011

(51) Int. Cl. A61K 9/48; A61K 9/20; A61K 36/82; A61P 39/06; A61K 36/63; A61K 36/9066; A61P 39/00; A61K 31/385; A61K 36/28

(52) U.S. Cl. 424/451; 514/440; 424/464; 424/729; 424/764; 424/769; 424/756

(57) ABSTRACT

Methods for using nutritional supplements containing lipoic acids and sulfur containing compounds to maintain or increase the levels of cellular GSH in the body are described. In some instances, the nutritional supplements can increase both the levels of GSH, vitamin C, GST activity, and antioxidant protection in the blood. The nutritional supplement contain a first part containing an effective amount of a lipoic acid and a second part containing an effective amount of a sulfur containing compound that increases the level of GSH vitamin C, GST activity, and antioxidant protection in the blood The first part and second part of the nutritional supplement can be partially or completely separated. The increased GSH and vitamin C levels increases the detoxification ability of the body as measured by the GST (Glutathione S-Transferases) activity and the antioxidant protection as measured by SAR (Serum Antioxidant Reserve). Other embodiments are described.

### FIELD

This application relates generally to methods for using nutritional supplements. More specifically, this application describes methods for using nutritional supplements containing lipoic acids, sulfur containing compounds, and other plant derived extracts for maintaining or increasing the levels of cellular glutathione in the body.

### BACKGROUND

Glutathione (GSH) is one of the most important antioxidants in the body. As such, maintenance of adequate levels of cellular GSH in the body is important. Several strategies have been proposed to maintain or increase the levels of cellular GSH. One strategy includes using nutritional supplements containing alpha lipoic acid (ALA), which is a potent antioxidant. While it is naturally present within cells, particularly in the mitochondria, orally delivered ALA can help increase the concentration in the cells. In addition to having an antioxidant effect in the cells, ALA has been shown to also increase levels of GSH.

Another strategy to maintain or increase the levels of cellular GSH includes using nutritional supplements containing cysteine, which is an amino acid and one of the biological building blocks of GSH. In certain conditions, it can be the rate limiting ingredient needed for the biosynthesis of GSH. Orally delivered forms of cysteine (including n-acetyl cysteine (NAC), cysteine hydrochloride, or other salts or derivatives of cysteine) have been shown to increase GSH and increase detoxification.

### SUMMARY

This application relates to methods for using nutritional supplements containing lipoic acids, sulfur containing compounds, and other plant-derived extracts to maintain or increase the levels of cellular GSH in the body. In some instances, the nutritional supplements can increase both the levels of cellular GSH and vitamin C in the blood. The nutritional supplement contains a first part containing an effective amount of a lipoic acid and a second part containing an effective amount of a sulfur containing compound that increase the level of cellular GSH. The first part and second part of the nutritional supplement can be partially or completely separated. The increased GSH and vitamin C levels increase both the detoxification ability of the body as measured by the GST (Glutathione S-Transferases) activity and antioxidant protection as measured by the SAR (Serum Antioxidant Reserve) assay.

### BRIEF DESCRIPTION OF THE DRAWINGS

The following description can be better understood in light of the Figures, in which:

FIG. 1 shows one chemical formula for alpha lipoic acid;

FIG. 2 shows one chemical formula for n-acetyl cysteine;

FIG. 3 shows the change in GSH levels in blood plasma over time resulting from administration of some embodiments of the nutritional supplements;

FIG. 4 shows the change in GST activity in blood plasma over time resulting from administration of some embodiments of the nutritional supplements;

FIG. 5 shows the change in SAR over time resulting from administration of some embodiments of the nutritional supplements; and

FIG. 6 shows the change in vitamin C levels in blood plasma over time resulting from administration of some embodiments of the nutritional supplements.

The Figures illustrate specific aspects of the nutritional supplements and methods for making and using such supplements. Together with the following description, the Figures demonstrate and explain the principles of the structures, methods, and principles described herein. In the drawings, the thickness and size of components may be exaggerated or otherwise modified for clarity. The same reference numerals in different drawings represent the same element, and thus their descriptions will not be repeated. Furthermore, well-known structures, materials, or operations are not shown or described in detail to avoid obscuring aspects of the described devices. Moreover, the Figures may show simplified or partial views, and the dimensions of elements in the Figures may be exaggerated or otherwise not in proportion for clarity.

### DETAILED DESCRIPTION

The following description supplies specific details in order to provide a thorough understanding. Nevertheless, the skilled artisan will understand that the nutritional supplements and associated methods of making and using such supplements can be implemented and used without employing these specific details. Indeed, the nutritional supplements and associated methods can be placed into practice by modifying the described supplements and methods and can be used in conjunction with any other apparatus and techniques conventionally used in the industry. For example, while description refers to nutritional supplements and associated methods for administration to a human, it could be modified and used in any mammal. As well, while the description refers to nutritional supplements, the compositions could be administered as dietary supplements or even as medications. In addition, where reference is made to a list of elements (e.g., elements a, b, c), such reference is intended to include any one of the listed elements by itself, any combination of less than all of the listed elements, and/or a combination of all of the listed elements.

Some embodiments of the nutritional supplements and methods for making and using such supplements are described herein and illustrated in the Figures. In these embodiments, the nutritional supplements contain a first part containing an effective amount of a lipoic acid (such as ALA) and a second part containing an effective amount of sulfur containing compounds (such as NAC) that increase the levels of cellular GSH and vitamin C. GSH and vitamin C play a major role in the overall antioxidant network and also act as an intermediate and/or conjugate in the detoxification process.

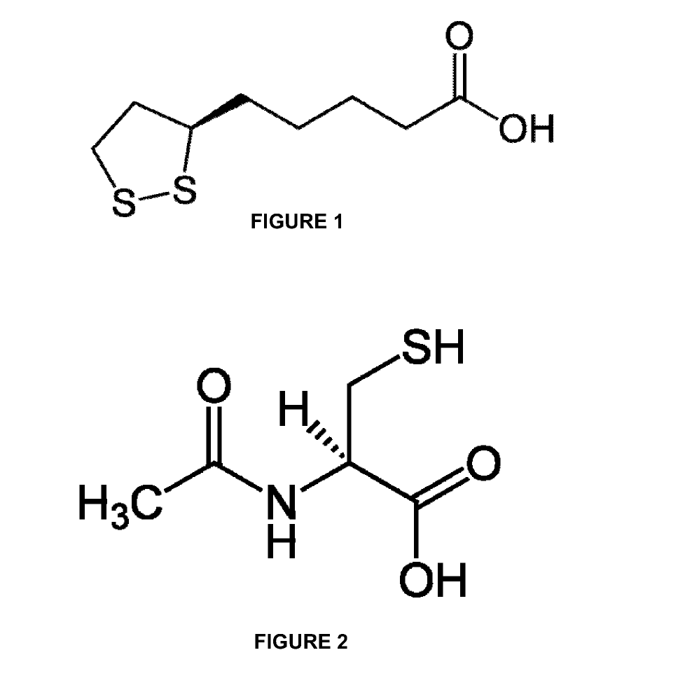

The nutritional supplements contain lipoic acids in any form. In some embodiments, the lipoic acid can be alpha lipoic acid (ALA), racemic alpha lipoic acid, di-hydro alpha lipoic acid, R-(+) alpha lipoic acid, S-(−) alpha lipoic acid, R-(+) dihydro alpha lipoic acid, S-(−) dihydro alpha lipoic acid, metal salts thereof, esters thereof, chelates thereof, precursors thereof, or combinations thereof. In other embodiments, the lipoic acid used in the nutritional supplement is alpha lipoic acid (ALA). One chemical formula of ALA is illustrated in FIG. 1.

The lipoic acid (i.e., ALA) can be contained in the nutritional supplement in any amount that, when combined with the sulfur containing compound, is able to maintain or increase the levels of GSH when it is administered. In some embodiments, the amount of lipoic acid can range up to 50 wt %. In other embodiments, the amount of lipoic acid can range from about 0.5 to about 20 wt %. In even other embodiments, the amount of lipoic acid can range from about 0.5 to about 7 wt %. In still other embodiments, the amount of lipoic acid can be any combination or sub-range of these amounts. With some tablet formulations, the amount of lipoic acid can range from about 5 mg/tablet to about 500 mg/tablet. With other tablet formulations, the amount of lipoic acid can range from about 40 mg/tablet to about 108 mg/tablet. With yet other tablet formulations, the amount of lipoic acid can range from about 60 mg/tablet to about 75 mg/tablet. In still other formulations, the amount of lipoic acid can be any combination or sub-range of these amounts.

The nutritional supplements also contain any sulfur containing compounds that are able to increase cellular GSH and vitamin C. In some embodiments, these sulfur containing compounds include cysteine, cystine, n-acetyl cysteine, broccoli, broccoli extract, or broccoli concentrate (sulforaphane), metal salts thereof, chelates thereof, precursors thereof, and combinations of these compounds. In some embodiments, these sulfur containing compounds comprise n-acetyl cysteine (NAC). One chemical formula of NAC is illustrated in FIG. 2. NAC is especially useful since it has been shown to be well absorbed by the intestine and is readily converted by the cells (particularly in the liver) to GSH. As well, NAC is especially useful when combined with ALA since ALA is known to mediate induction of GSH and NAC is a building block of GSH; therefore these two ingredients work in concert and perhaps synergistically.

These sulfur containing compounds can be contained in the nutritional supplement in any amount that, when combined with the lipoic acid compound, is able to maintain or increase the levels of GSH and vitamin C when it is administered. In some embodiments, the amount of the sulfur containing compounds can range up to 50 wt %. In other embodiments, the amount of the sulfur containing compounds can range from about 0.5 to about 20 wt %. In even other embodiments, the amount of the sulfur containing compounds can range from about 0.5 to about 7 wt %. In still other embodiments, the amount of the sulfur containing compounds can be any combination or sub-range of these amounts. With some tablet formulations, the amount of the sulfur containing compounds can range from about 5 mg/tablet to about 500 mg/tablet. With other tablet formulations, the amount of lipoic acid can range from about 40 mg/tablet to about 108 mg/tablet. With yet other tablet formulations, the amount of lipoic acid can range from about 60 mg/tablet to about 75 mg/tablet.

Besides the lipoic acid and sulfur containing compounds, the nutritional supplement (NS) can also contain any other known dietary or nutritional compounds that can be added to either (or both) parts of the NS, including those described in U.S. application Ser. No. 13/196,390, the entire disclosure of which is incorporated herein by reference. For example, the NS can also contain phenolic antioxidants, such as—but not limited to—milk thistle, green tea, olive extracts, turmeric extracts, other plant derived phenolic extracts, and combinations thereof. The NS can also contain other additives, such as binders, disintegrants, lubricants, flowing agents, flavorings, coatings, and combinations of these additives that can be added to either (or both) parts of the NS, as described in U.S. application Ser. No. 13/196,390, the entire disclosure of which is incorporated herein by reference. The NS can be prepared and delivered in various forms, including tablets, capsules, and powders, as described in U.S. application Ser. No. 13/196,390, the entire disclosure of which is incorporated herein by reference.

Based on their individual abilities to increase GSH and vitamin C levels, a nutritional supplement containing a combination of ALA and NAC should also increase cellular GSH and vitamin C levels. However, these ingredients cannot be effectively combined with each other in a single nutritional supplement because they react with each other when they come into contact, thereby becoming destabilized and decreasing the efficacy of the product. With the nutritional supplements described herein, these two ingredients (ALA and NAC) can be combined into a single delivery vehicle that limits the contact between the two ingredients as described in U.S. application Ser. No. 13/196,390, the entire disclosure of which is incorporated herein by reference. In such configurations, high levels of both of these ingredients can be delivered (and maintained) in a single oral dosage, increasing the levels of GSH and providing a detoxification system without the ALA and NAC becoming destabilized.

The NS can be used as a dietary enhancement as known in the art by administration to a human or an animal. The method of administration will depend on the form of the nutritional supplement. In the instances where the NS is in the form of a powder, the powder can be added to food or liquids that are ingested by an individual. In the instances where the NS is in the form of a tablet or capsule, the tablet/capsule can be added to solid food (or liquids) that is ingested by an individual, or the tablet/capsule can be ingested by itself.

The amount of the NS that needs to be ingested by an individual—or the recommended dose—will depend on numerous factors, including the weight, height and age of an individual, the metabolism of an individual, the health status, ethnicity, genetics, environment, and/or lifestyle. In some embodiments, the amount of NS ingested can range up to about 5.4 g in a day. In other embodiments, the amount of NS ingested can range from about 0.95 g to about 2.85 g in a day. In still other embodiments, the dosage can be any combination or sub-range of these amounts. For example, for an individual with normal health and a weight range of about 130 to about 190 lbs, the recommended amount of NS can be about 2.85 g/day.

The NS can be administered in a single dose or in multiple doses within a set period of time (usually a 24 hour period). In some embodiments, the NS can be administered in a single dose when the concentration of the active ingredients is high enough. In other embodiments, the NS can be administered in several dosages (i.e., 2, 3, 4, etc.) over the same period of time. For example, for an individual with normal health and a weight range of about 130 to about 190 lbs, the recommend administration is 3 times/day.

In some embodiments, the NS can maintain or increase the concentration of GSH within the blood of the body. GSH is an abundant low-molecular weight, water soluble antioxidant in our bodies and plays a major role in the detoxification process. Unfortunately, GSH is not typically efficiently absorbed from the diet and must be synthesized in the body by specific Phase II enzymes. Phase I detoxification enzymes contain the cytochrome P450 family of enzymes and serve as the first line of defense to initially oxidize toxins. Unfortunately, this initial oxidation can convert compounds (e.g., benzo(a)pyrene, etc.) into more toxic metabolites. Phase II enzymes, however, can further modify Phase I metabolites by conjugating them with water soluble molecules. This modification renders them less toxic and as a result, more easily excreted from the body. If not modified by Phase II enzymes, Phase I metabolites can damage biomolecules (e.g., DNA, proteins, lipids, etc.); therefore, it is helpful that this detoxification system continually favors Phase II reactions. And the levels and efficiency of Phase II detoxification enzymes, as well as the pool of conjugation substrates, should remain high to maintain optimal liver detoxification capacity. Two important antioxidant and conjugation substrates involved in these detoxification pathways are GSH and vitamin C.

Under normal conditions, GSH production by the body is at steady-state. Thus, GSH is normally present at concentrations of about 10 mM inside living cells. During times of toxicological insult, however, the body increases production of GSH by up-regulating this Phase II enzymatic machinery. Both sulfur-containing compounds (including broccoli extract and NAC) and alpha-lipoic acid have also been shown to upregulate Phase II enzymes, including those that synthesize GSH. Thus, the nutritional supplements described herein can increase the concentration of GSH in living cells. In some embodiments, administering the nutritional supplements described herein can increase the total GSH concentration in the blood up to about 100%. In other embodiments, administering the nutritional supplements can increase the GSH concentration from about 5% to about 50%. In yet other embodiments, administering the nutritional supplements can increase the GSH concentration from about 20% to about 30%. In still other embodiments, the concentration of GSH can be any combination or sub-range of these amounts.

In some embodiments, administering the NS can increase the concentration of vitamin C within the blood of the body. Vitamin C can be an important line of defense in protecting biomolecules from oxidative damage, especially in the circulation of blood. Moreover, some recent scientific evidence suggests that vitamin C may also play a role in the removal of toxins. However, the concentration of vitamin C in the body is tightly regulated by intestinal absorption from the diet and recycling by the kidneys. Because of this tight regulation, it was previously thought that circulating vitamin C levels are relatively fixed and could only be increased by supplementing with vitamin C. However, it was recently shown that both circulating and tissue levels of vitamin C can be increased by certain phytochemicals (such as lipoic acid) even in the absence of vitamin C supplementation.

In some embodiments, administering the nutritional supplements described herein can increase the vitamin C concentration in the blood up to about 75%. In yet other embodiments, administering the nutritional supplements can increase the vitamin C concentration from about 10% to about 50%. In yet other embodiments, administering the nutritional supplements can increase the vitamin C concentration from about 20% to about 30%. In still other embodiments, the increase of vitamin C can be any combination or sub-range of these amounts.

In some embodiments, administering the NS can increase the concentration of both GSH and vitamin C within the blood of the body. As noted above, both GSH and vitamin C are molecules that can be utilized for both antioxidant defenses and substrates for endogenous detoxification reactions. In some embodiments, administering the nutritional supplements can increase the vitamin C concentration in the blood up to about 75% while also increasing the level of GSH concentration up to about 100%. In other embodiments, administering the nutritional supplements can increase the vitamin C concentration from about 10% to about 50% while also increasing the level of GSH concentration from about 5% to about 50%. In yet other embodiments, administering the nutritional supplements can increase the vitamin C concentration from about 20% to about 30% while also increasing the level of GSH concentration from about 20% to about 30%. In still other embodiments, the increase of vitamin C and GSH can be any combination or sub-range of these amounts.

By increasing the GSH and vitamin C concentration, the NS can also increase the detoxification and antioxidant ability of the body. This GSH-dependent detoxification ability can be measured by GST activity. GSTs are a class of enzymes that catalyze the conjugation of reduced GSH, via the sulfhydryl group, to various electrophilic compounds. This activity assists in the removal and excretion of a number of compounds including oxidized biomolecules, toxins, and other xenobiotics. Serum Antioxidant Reserve (SAR) can measure the antioxidant capacity of human blood. The diet supplementation with the NS described herein can encourage an increase in circulating GSH; thus, total GST activity can serve as a functional detoxification endpoint. A corresponding increase in SAR can confirm that an individual's blood experienced increased protection from oxidative stress, thereby proving the effectiveness of the NS in increasing overall antioxidant status.

In some embodiments, administering the nutritional supplements can increase the SAR level in the blood up to about 150%. In other embodiments, administering the nutritional supplements can increase the SAR level from about 20% to about 75%. In yet other embodiments, administering the nutritional supplements can increase the SAR level from about 20% to about 60%. In still other embodiments, the increase of SAR can be any combination or sub-range of these amounts.

In some embodiments, administering the nutritional supplements can increase the GST (Glutathione S-Transferases) activity in the blood. GSTs are a class of enzymes that can catalyze the conjugation of GSH to various toxins. Therefore, the measurement of GST activity can be indicative of the body's phase II detoxification capacity. It is believed that an increase in GSH after supplementation with the NS would improve GST-facilitated detoxification. It is also believed that a simultaneous increase in GSTs activity would indicate that not only is there more substrate (GSH) present in the body, but that the substrate is actually being conjugated to toxins at a greater rate.

In some embodiments, administering the nutritional supplements can increase the GST activity in the blood up to about 50%. In other embodiments, administering the nutritional supplements can increase the GST activity from about 1% to about 20%. In yet other embodiments, administering the nutritional supplements can increase the GST activity from about 5% to about 15%. In still other embodiments, the increase of GST activity can be any combination or sub-range of these amounts.

Decreased GSH levels can be associated with a wide variety of conditions including: pathophysiological conditions, including hepatic dysfunction, malignancies, HIV infection, pulmonary disease, and Parkinson's disease; immunologic illnesses such as asthma or arthritis and chronic diseases, such as diabetes or cardiovascular disease. Low GSH levels have also been implicated in the normal aging process. Indeed, sustained GSH depletion levels can be fatal within humans in certain conditions such as heavy metal, mushroom, or acetaminophen overdose. Thus, in some embodiments, administering the NS to maintain or increase the concentration of GSH can also treat these conditions or illnesses.

The Example below illustrates the methods for using nutritional supplements containing lipoic acids and sulfur containing compounds.

### Example 1

Several packaged tablets were manufactured to contain the ingredients shown in Table 1 in a daily dose of 3 tablets using the methods described in U.S. application Ser. No. 13/196,390, the entire disclosure of which is incorporated herein by reference.

TABLE 1

| INGREDIENT | AMOUNT |
|---|---|
| CHOLINE BITARTRATE | 375 mg |
| MILK THISTLE EXTRACT (SILYBUM MARIANUM L., | 240 mg |
| FRUIT, STANDARDIZED TO CONTAIN 80% SILIMARIN | |
| BY UV) | |
| N-ACETYL L-CYSTEINE | 225 mg |
| ALPHA-LIPOIC ACID | 200 mg |
| BROCCOLI CONCENTRATE (BRASSICA OLERACEA V. | 75 mg |
| BOTRYTIS L., FLOWER) | |
| GREEN TEA EXTRACT-DECAFFEINATED (CAMELLIA | 45 mg |
| SINENSIS HUNT, LEAVES) | |
| OLIVOL ® (OLIVE EXTRACT, OLEA EUROPAEA L., | 45 mg |
| FRUIT) | |
| TURMERIC EXTRACT (CURCUMA LONGA L., ROOT) | 45 mg |
| BIOTIN | 225 μg |
| MICROCRYSTALLINE CELLULOSE | Trace |
| ASCORBYL PALMITATE | Trace |
| PREGELATINIZED STARCH | Trace |
| SILICON DIOXIDE | Trace |
| CROSCARMELLOSE SODIUM | Trace |
| MODIFIED CELLULOSE | Trace |
| DEXTRIN | Trace |
| DEXTROSE | Trace |
| LECITHIN | Trace |
| NATURAL FLAVOR | Trace |
| SODIUM CITRATE | Trace |

Fifteen healthy individuals between the ages of 25 and 45 were selected. The tablets were administered to seven of these individuals in split doses from three tablets to match the total amount of nutrients listed in Table 1 along with a specified diet. The other eight individuals (the placebo group) received placebo tablets with a substantially similar diet. Subjects continued to take the active or placebo tablets for a total of 28 days.

During the acute phase of the study, the effects of this nutritional supplement were monitored during the first eight hours following the initial treatment (i.e., day 1, hour 0). During the chronic phase of the study, the effects of the nutritional supplement relative to baseline GSH and vitamin C levels after 28 days of administration were measured. During the acute-on-chronic phase of the study, any acute effects that occurred in addition to the 28 day chronic phase were monitored.

### GSH Levels

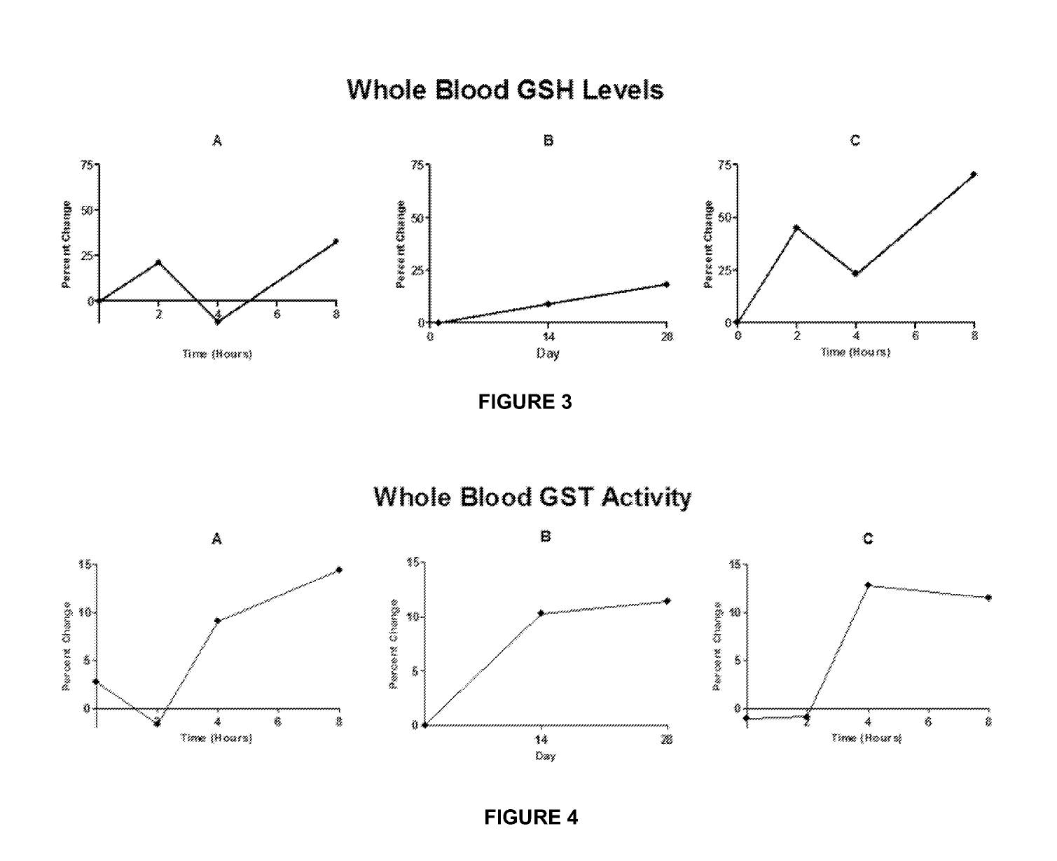

The amount of GSH in blood plasma was measured in the following manner. GSH analytes were separated out by injecting 1 μL of the prepared plasma sample into an Agilent (Series 1200) HPLC using a Phenomenex C₁₈ column. The method conditions used during the measurement were 0-7 minutes at 0.5 mL/min 0.03% formic acid in water and 8-15 minutes 0.5 mL/min 2-propanol. Both the reduced glutathione (GSH) and oxidized glutathione (GSSG) were detected on an Agilent tandem mass spectrometer (Series 6410, Model G6410A) using an electrospray (ES) source. The glutathione concentrations were determined relative to authentic standards and expressed as total soluble glutathione (GSH+2GSSG) relative to the placebo group and reported in FIG. 3. Section A of FIG. 3 shows the glutathione concentrations in acute phases, section B shows the glutathione concentrations in chronic phases, and section C shows the glutathione concentrations in acute-on-chronic phases.

As shown in FIG. 3, this nutritional supplement tablet increased plasma GSH 2 hours following the first treatment and significantly increased plasma GSH 8 hours after supplementation. As well, a chronic (18.3%) increase in plasma GSH was observed, but did not reach statistical significance. This nutritional supplement significantly increased plasma GSH 8 hours after supplementation, during the acute-on-chronic phase. And plasma GSH levels increased 74% by administration of the nutritional supplement.

These results show that circulating GSH levels increased following the treatment regimen with this nutritional supplements. These results also show a biphasic response in both the acute and acute-on-chronic phases of the study, especially FIG. 3. Without being limited to this explanation, it is believed that this result can be explained by two distinct mechanisms. The first mechanism is likely due to the n-acetyl-L-cysteine used in the nutritional supplements. Cysteine is the rate-limiting substrate in GSH synthesis and providing cysteine alone can increase GSH levels. As such, a concurrent significant increase in plasma cysteine levels following treatment with these nutritional supplements was found. The second mechanism is likely due to an increase in Phase II GSH-synthesizing enzymes over time. Alpha-lipoic acid, some sulfur-containing compounds, and other plant derived extracts (i.e., milk thistle, broccoli extract, etc.) have been shown to up-regulate Phase II enzymes and likely account for the steady increase in GSH observed during this study. The large acute-on-chronic increase in GSH, relative to the acute phase, is likely due to this steady increase in GSH in addition to the effects seen during the acute phase.

### GST Activity

The GST activity in the blood plasma was measured in the following manner. Whole blood samples from the individuals were collected and flash-frozen in liquid nitrogen before storage at −120° C. The stored samples were then thawed at room temperature before analysis. A small aliquot of whole blood was diluted and assayed for GST activity using a glutathione S-Transferase Assay Kit (#703302 Cayman Chemical, Ann Arbor, Mich.) according to the manufacturer instructions and reported in FIG. 4. Section A of FIG. 4 shows the GST activity in acute phases, section B shows the GST activity in chronic phases, and section C shows the GST activity in acute-on-chronic phases.

As shown in FIG. 4, this nutritional supplement acutely, chronically, and acute-on-chronically increased whole blood GST activity. As well, this nutritional supplement significantly increased whole blood GST activity 8 hours after administration during the acute and acute-on-chronic phase (p<0.05). And a chronic 11.4% increase in GST activity was observed, but did not reach statistical significance.

### SAR Level

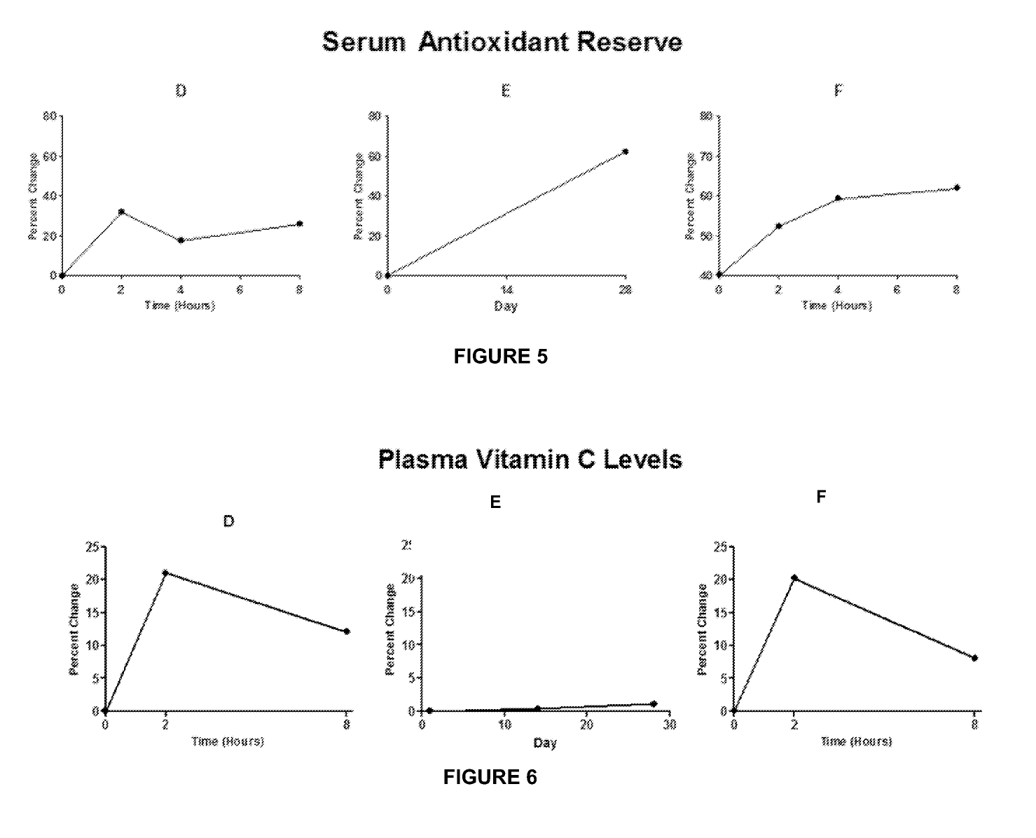

The SAR (serum antioxidant reserve) was measured in the following manner. Blood was collected in a 7.5 ml SST Vacutainer tube (BD, 1 Becton Drive Franklin Lakes, N.J. USA 07417), allowed to clot for 30 minutes, and then centrifuged at 1400*g for 12 minutes to obtain serum. Oxidation of serum was initiated by addition of 1.0 mmol SIN-1 chloride. After incubation for 4 hours at 37° C., the oxidation reaction was stopped by addition of BHT and EDTA. The induced 8-isoprostanes was then measured using an ELISA kit (8-iso Prostaglandin F₂ Kit. Cayman Chemical, Ann Arbor, Mich.). Calibration, curve fitting, and data analysis was conducted according to the manufacturer instructions. The changes in SAR over time are reported in FIG. 5. Section A of FIG. 4 shows the SAR change in acute phases, section B shows the SAR change in chronic phases, and section C shows the SAR change in acute-on-chronic phases.

As shown in FIG. 5, administration of this nutritional supplement significantly increased SAR relative to the control group by 32% within 2 hours following the first treatment. There was a significant increase for all acute time points measured (t=2, 4, 8 h; p<0.05). A chronic (40%) increase in SAR was seen by the 28^{th} day and was statistically significant (p<0.05). This nutritional supplement significantly increased SAR (by 21%) 8 hours after administration during the acute-on-chronic phase (p<0.05). Overall, daily administration of this nutritional supplement increased SAR by 62%.

These results show that the recommended dosage of these nutritional supplements, taken regularly, can increase both GST activity and SAR resulting and leading to increased detoxification and antioxidant capacity. The combination of ingredients in the nutritional supplement (including broccoli extract, milk thistle, NAC, alpha-lipoic acid, etc.), not only increased plasma GSH levels simultaneously, but it is believed that they also upregulated the molecular mechanism needed to utilize GSH in the detoxification reactions and significantly increase the overall capacity of the blood to protect against oxidative insult.

### Vitamin C Levels

The level of vitamin C in the blood plasma was measured in the following manner. vitamin C was separated by injecting 7 μL of the prepared sample of the blood sample into an Agilent Series 1200 HPLC using an Agilent C₁₈ column. The conditions of the HPLC column were 0-5 mL/min of 0.03% formic acid in water and 8-15 minutes of 0.5 mL/min 2-propanol. The vitamin C concentration was detected using a Hewlett-Packard UV detector (254 nM; Series 1050) and were determined relative to authentic standards. The changes in vitamin C over time are reported in FIG. 6. Section A of FIG. 4 shows the vitamin C change in acute phases, section B shows the vitamin C change in chronic phases, and section C shows the vitamin C change in acute-on-chronic phases.

As shown in section A of FIG. 6, administration of this nutritional supplement increased plasma vitamin C as soon as two 2 hours following the first treatment and was maintained during the entire acute phase (0-8 hour time points; p<0.05). As shown in section B of FIG. 6, this nutritional supplement also results in a chronic increase in vitamin C, but it did not reach statistical significance. And as shown in section C of FIG. 6, administration of this nutritional supplement yielded significantly higher plasma vitamin C concentrations during the acute-on-chronic phase of the study (p<0.05).

The increase found in vitamin C levels is unexpected because the nutritional supplement does not contain vitamin C. Vitamin C is a well known antioxidant and is known to act as an important substrate for detoxification reactions in the body since recent studies have shown that vitamin C binds to, and likely facilitates, the removal of toxins. These results support the recent finding that supplementation with phytochemicals, such as ALA, can increase vitamin C transporter levels and can also increase vitamin C levels in the absence of vitamin C supplementation. And in these results, the subjects were neither taking any nutritional supplements prior to enrollment in the study nor was there vitamin C in the provided meal that was administered to the subjects.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method for maintaining or increasing the levels of glutathione in the blood, comprising administering a nutritional supplement to a mammal in need thereof, the nutritional supplement containing:
a first part containing an effective amount of a lipoic acid; and
a second part containing an effective amount of a sulfur containing compound that increases the level of cellular glutathione;
wherein the first part and second part are partially or completely separated.

2. The method of claim 1, wherein the lipoic acid comprises alpha lipoic acid.

3. The method of claim 1, wherein the sulfur containing compound comprises n-acetyl cysteine.

4. The method of claim 1, wherein the nutritional supplement is in the form of a tablet or capsule with the first part forming a first layer and the second part forming a second layer.

5. The method of claim 1, wherein the level of glutathione is increased by up to about 100%.

6. The method of claim 5, wherein the level of glutathione is increased by about 5 to about 50%.

7. The method of claim 1, further comprising increasing the level of vitamin C in the blood.

8. The method of claim 7, wherein the level of vitamin C is increased by up to about 75%.

9. The method of claim 7, wherein the level of vitamin C is increased by about 10% to 50%.

10. A method for increasing the level of glutathione and vitamin C in the blood, comprising administering a nutritional supplement to a mammal in need thereof, the nutritional supplement containing:
a first part containing an effective amount of a lipoic acid; and
a second part containing an effective amount of a sulfur containing compound that increases the level of cellular glutathione;
wherein the first part and second part are partially or completely separated.

11. The method of claim 10, wherein the lipoic acid comprises alpha lipoic acid.

12. The method of claim 10, wherein the sulfur containing compound comprises n-acetyl cysteine.

13. The method of claim 10, wherein the nutritional supplement is in the form of a tablet or capsule with the first part forming a first layer and the second part forming a second layer.

14. The method of claim 10, wherein the level of glutathione is increased by up to about 100%.

15. The method of claim 14, wherein the level of glutathione is increased by about 5 to about 50%.

16. The method of claim 10, wherein the level of vitamin C is increased by up to 75%.

17. The method of claim 16, wherein the level of vitamin C is increased by about 10% to about 50%

18. A method for detoxifying a mammal, comprising administering a nutritional supplement to a mammal in need thereof, the nutritional supplement containing:
a first part containing an effective amount of a lipoic acid; and
a second part containing an effective amount of a sulfur containing compound that increases the level of cellular glutathione;
wherein the first part and second part are partially or completely separated.

19. The method of claim 18, wherein the lipoic acid comprises alpha lipoic acid and the sulfur containing compound comprises n-acetyl cysteine.

20. The method of claim 18, wherein the nutritional supplement is in the form of a tablet or capsule with the first part forming a first layer and the second part forming a second layer.

21. The method of claim 18, wherein the GST activity is increased by up to about 50%.

22. The method of claim 21, wherein the GST activity is increased by about 1% to about 20%.

23. A method for increasing the antioxidant protection of a mammal, comprising administering a nutritional supplement to a mammal in need thereof, the nutritional supplement containing:
a first part containing an effective amount of a lipoic acid; and
a second part containing an effective amount of a sulfur containing compound that increases the level of cellular glutathione;
wherein the first part and second part are partially or completely separated.

24. The method of claim 23, wherein the lipoic acid comprises alpha lipoic acid and the sulfur containing compound comprises n-acetyl cysteine.

25. The method of claim 23, wherein the nutritional supplement is in the form of a tablet or capsule with the first part forming a first layer and the second part forming a second layer.

26. The method of claim 23, wherein the SAR is increased by up to about 150%.

27. The method of claim 26, wherein the SAR is increased by about 20 to about 75%.

28. The method of claim 1, wherein the first or second part further comprises milk thistle, green tea, olive extracts, turmeric extracts, or other plant derived phenolic extracts.

29. The method of claim 10, wherein the first or second part further comprises milk thistle, green tea, olive extracts, turmeric extracts, or other plant derived phenolic extracts.

30. The method of claim 18, wherein the first or second part further comprises milk thistle, green tea, olive extracts, turmeric extracts, or other plant derived phenolic extracts.

31. The method of claim 23, wherein the first or second part further comprises milk thistle, green tea, olive extracts, turmeric extracts, or other plant derived phenolic extracts.
